(19) United States
(12) Patent Application Publication
(10) Pub. No.: US 2020/0165659 A1
(43) Pub. Date: May 28, 2020

(54) METHOD FOR IDENTIFICATION OF THE GLIDING BEHAVIOUR OF STENOTROPHOMONAS MALTOPHILIA, AND ASSOCIATED ANTIBIOTICS PRODUCTION THEREOF

(71) Applicant: Yunxuan Xie, Tianjin (CN)

(72) Inventor: Yunxuan Xie, Tianjin (CN)

(21) Appl. No.: 16/201,120

(22) Filed: Nov. 27, 2018

(51) Int. Cl. C12Q 1/24; C12Q 1/689; C12N 1/20

(52) U.S. Cl. CPC C12Q 1/24; C12N 1/20; C12Q 1/689

(57) ABSTRACT

A method for identification of the gliding behaviour of Stenotrophomonas maltophilia, and associated metabolites production. The manipulation of carbon sources comprising glucose, galactose, glycerol and sucrose and nitrogen sources comprising tryptone, bacterial peptone, yeast extract and ammonium chloride at a concentration (w/v) of 0.1%, 0.5%, 1% and 2% in the culture medium dictated the change in the gliding behaviour of S. maltophilia and metabolite production in-vivo. A diversified metabolic profile is observed when gliding motility is restricted and no metabolite is produced when gliding motility is observed in S. maltophilia. The culture medium supplemented with 1˜8% glycerol as the carbon source and 0.1˜0.8% yeast extract as the nitrogen source resulted in the identification of two major metabolites bearing anti-Staphylococcus aureus activity in the crude extract (5 μl) of the Stenotrophomonas maltophilia, strain P3-15.

### REFERENCE TO A SEQUENCE LISTING

This application contains a Sequence Listing in computer readable form. The computer readable form is incorporated herein by reference.

### BACKGROUND OF THE INVENTION

### Field of the Invention

The invention generally relates to identification of metabolite production based on gliding behavior of bacteria and more particularly, relates to a method for identification of the gliding behavior of Stenotrophomonas maltophilia, strain P3-15 in different culture conditions and its associated metabolites production.

### Description of the Related Art

Isolating new bacterial strains has been a consistent effort for natural product discovery. Mining novel metabolites from gliding gram-negative bacteria has been increasingly appreciated in recent years (Newman, D. J., Cragg, G. M., 2012. Natural products as sources of new drugs over the 30 years from 1981 to 2010. J. Nat. Prod. 75(3), 311-335; Koehn, F. E., Carter, G. T., 2005. The evolving role of natural products in drug discovery. Nat. Rev. Drug. Discov. 4(3), 206-220). Representative strains include cyanobacteria, Myxococcus and Lysobacter strains (Walsh, C. T., Fischbach, M. A., 2010. Natural products version 2.0: connecting genesto molecules. J. Am. Chem. Soc. 132(8), 2469-2493; Bush, K., Courvalin, P., Dantas, G., Davies, J., Eisenstein, B., Huovinen, P., Jacoby, G. A., Kishony, R., Kreiswirth, B. N., Kutter, E., Lerner, S. A., Levy, S., Lewis, K., Lomovskaya, O., Miller, J. H., Mobashery, S., Piddock, L. J., Projan, S., Thomas, C. M., Tomasz, A., Tulkens, P. M., Walsh, T. R., Watson, J. D., Witkowski, J., Witte, W., Wright, G., Yeh, P., Zgurskaya, H. I., 2011. Tackling antibiotic resistance. Nat. Rev. Microbiol. 9, 894-896). Compounds that were isolated from these strains and put in line of clinical trial include anti-tumor Curacin A, Epothilone and Dolastatin, suggesting the potential of gram-negative strains to be developed into novel antibiotic producer (Verdier-Pinard, P., Lai, J., Yoo, H., Yu, J., Marquez, B., Nagle, D., Nambu, M., White, J., Falck, J., Gerwick, W., Day, B., Hamel, E., 1998). Structure-activity analysis of the interaction of curacin A, the potent colchicine site antimitotic agent, with tubulin and effects of analogs on the growth of MCF-7 breast cancer cells (Mol. Pharmacol. 53(1), 62-76; Aguilar, N., Kruger, J., 2002). Toward a library synthesis of the natural dipeptide antibiotic TAN 1057 A, B (Molecules. 7(6), 469-474). In recent years, Lysobacter genus in Xanthomonadaceae family was discovered to produce a variety of natural products with broad scope biological activities (Xie, Y., Wright, S., Shen, Y., Du, L., 2012). Bioactive natural products from Lysobacter (Nat. Prod. Rep. 29(11), 1277-1287). Representative compounds include antifungal HSAF, antibacterial lysobactin, anti-MRSA WAP and tripropeptin. With regard to the number of compounds isolated from these strains, genomic potential of synthesizing these natural products was huge (Wenzel, S. C., Muller, R., 2009). Myxobacteria—‘microbial factories’ for the production of bioactive secondary metabolites. Mol. Biosyst. 5(6), 567-574).

Stenotrophomonas maltophilia is a gram-negative bacterium with close phylogenetic relationship to Lysobacter, and was initially discovered and identified by Hugh and Ryschenkow (Hugh, R., Ryschenkow, E., 1961. Pseudomonas maltophilia, an alcaligenes-like Species. J. gen. Microbiol. 26, 123-132). Stenotrophomonas maltophilia is distributed among various ecological niches and was featured by their rapid duplication rates under laboratory culture condition. Unlike the well-studied Lysobacter, efforts to identify natural products from S. maltophilia have been limited to characterization of potential biological activities from bacterial crude extract (Jakobi, M., Winkelmann, G., Kaiser, D., Kempler, C., Jung, G., Berg, G., Bahl, H., 1996. Maltophilin: a new antifungal compound produced by Stenotrophomonas maltophilia R3089. J. Antibiot. 49(11), 1101-1104). With the completion of multiple genome mining projects of S. maltophilia, biosynthetic gene clusters of a series of secondary metabolites were unraveled (S. maltophilia 13637 (Accession: CP008838.1) and S. maltophilia K279a (Accession: NC_010943.1)). Despite that few natural products were identified from S. maltophilia, the genomic potential to biosynthesize these natural products is huge.

Reported studies for mining bacterial species to remove benthic organic contaminants in a polluted river suggested the enrichment of bacterial abundance within benthic soil niche by the addition of calcium nitrate (Wang, L. H., Zheng, L. V., Hao, C. B., Wang, G. C., & Shi, P. (2013)). Degrading bacteria community structure in groundwater of a petroleum-contaminated site (Environmental Science & Technology, 36(7), 1-45)). Enrichment bacterial species include Pseudoxanthomonas, Lysobacter and Simplicispira, the first two of which belonged to Xanthomonas family.

Frequently used solid culture medium for Xanthomonas selection mostly contains regular carbon sources (such as glucose), a mixed animal source or bacterial source nitrogen (such as peptone and yeast extract) and combines essential metal elements in zwitter ionic form. This culture method facilitates the growth of most culturable bacterial species but does not favor the accumulation of Xanthomonas strains in particular (Bae, H. S., Im, W. T., & Lee, S. T. (2005)). Lysobacter concretionis sp. nov. isolated from anaerobic granules in an upflow anaerobic sludge blanket reactor (International Journal of Systematic & Evolutionary Microbiology, 55(Pt 3), 1155.). In relatively limited studies, special carbon and nitrogen sources for Xanthomonas enrichment were used (Romanenko, L. A., Uchino, M., Tanaka, N., Frolova, G. M., & Mikhailov, V. V. (2008). Lysobacter spongiicola sp. nov. isolated from a deep-sea sponge. International Journal of Systematic & Evolutionary Microbiology, 58(Pt 2), 370). Samples collected from seawater were subject to a special medium selection using 3-[N-tris(hydroxymethyl)-methylamino]-2-hydroxypropanesulfonic acid. These enrichment methods, however, do not ensure the growth advantage of Xanthomonas species over the others, especially the fast growing E. coli species and lead to the simultaneous proliferation of many other types of bacteria. To allow the specific enrichment of Xanthomonas strains, attempts were made to use special carbon and nitrogen source using radiolabelled substrates (Gallagher, E. M., Young, L. Y., Mcguinness, L. M., & Kerkhof, L. J. (2010)). Detection of 2,4,6-trinitrotoluene-utilizing anaerobic bacteria by 15n and 13c incorporation (Applied & Environmental Microbiology, 76(5), 1695-8). These attempts enriched some Xanthomonas strains at moderate efficiency.

SmeD gene is the major quinolone resistance determinant that encodes for an efflux pump for antibiotic repulsion and is primarily present within many resistant Xanthomonas family members (Sanchez, P., Le, U., & Martinez, J. L. (2003)). The efflux pump inhibitor Phe-Arg-β-naphthylamide does not abolish the activity of the Stenotrophomonas maltophilia SmeDEF multidrug efflux pump (Journal of Antimicrobial Chemotherapy, 51(4), 1042.; Huang, Y. W., Lin, C. W., Ning, H. C., Lin, Y. T., Chang, Y. C., & Yang, T. C. (2017)). Overexpression of SmeDEF efflux pump decreases aminoglycoside resistance in Stenotrophomonas maltophilia. Antimicrobial Agents & Chemotherapy, 61(5), AAC.02685-16.). A conserved sequence of 150 bp was believed to be specific for S. maltophilia strains (Alonso and Martinez, A molecular biological protocol to distinguish potentially human pathogenic Stenotrophomonas maltophilia from plant-associated Stenotrophomonas rhizophila. Environmental Microbiology (2005)7(11), 1853-1858).

Type IV pili-dependent gliding motility has been detected in many Gram-negative bacteria but not in Gram-positive bacteria and was considered to be essential for gliding bacteria (John J. Varga, Van Nguyen, David K. O'Brien, Katherine Rodgers, Richard A. Walker and Stephen B. Melville, Type IV pili-dependent gliding motility in the Gram-positive pathogen Clostridium perfringens and other Clostridia. Molecular Microbiology (2006) 62(3), 680-694). Among many annotated genome sequences, the pilus biosynthetic gene clusters were identified (Gene ID: 31883789, 878186). Initial gliding behavior of gram-negative bacteria was featured by the appearance of rugged periphery, suggesting that bacterial cells were moving in individual directions at different rates (John J. Varga, Van Nguyen, David K. O'Brien, Katherine Rodgers, Richard A. Walker, Stephen B. Melville. Molecular Microbiology (2006) 62(3), 680-694). Type IV pili-dependent gliding motility in the Gram-positive pathogen Clostridium perfringens and other Clostridia). Several studies in Gram-negative gliding bacteria suggest that gliding behaviour was controlled by complicated intracellular mechanisms initiated with the external medium stimuli (Murray, T., & Kazmierczak, B. (2008). Pseudomonas aeruginosa exhibits sliding motility in the absence of type IV pili and flagella. Journal of Bacteriology, 190(8), 2700-2708.). L. enzymogenes, a Gram-negative strain with close phylogenetic relationship to S. maltophilia, was found to attach to fungal hypha that was dependent on type IV pilus (T4P) when confronting the infection of Fusarium verticilloides. However, this type of movement is not visible unless being examined under microscope (Mathioni S M, Patel N, Riddick B, Sweigard J A, Czymmek K J, Caplan J L, Kunjeti S G, Kunjeti S, Raman V, Hillman B I, Kobayashi D Y, Donofrio N M. Transcriptomics of the rice blast fungus Magnaporthe oryzae in response to the bacterial antagonist Lysobacter enzymogenes reveals candidate fungal defense response genes. PLoS ONE. 2013; 8(10):e76487; Patel N, Cornejo M, Lambert D, Craig A, Hillman B I, Kobayashi D Y. A multifunctional role for the type IV pilus in the bacterial biological control agent Lysobacter enzymogenes. Phytopathology. 2011; 101(6): S138). Several Gram-negative strains have T4P as a hair-like appendage on the surface and carries out various functions such as surface motility, host attachment and pathogenesis (Burdman S, Bahar O, Parker J K, De La Fuente L. Involvement of type IV pili in pathogenicity of plant pathogenic bacteria. Genes. 2011; 2(4):706-735; Mattick J S. Type IV pili and twitching motility. Annu Rev Microbiol. 2002; 56:289-314; Patel N, Cornejo M, Lambert D, Craig A, Hillman B I, Kobayashi D Y. A multifunctional role for the type IV pilus in the bacterial biological control agent Lysobacter enzymogenes. Phytopathology. 2011; 101(6):S138).

Gram-negative motile predators are potential producers of multiple secondary metabolites and usually upon nutrient depletion, undergo concerted cellular differentiation to form particular cell morphologies for efficient nutrient trapping (usually mediated by highly polymeric extracellular polysaccharides). The ecological significance of EPS includes the formation of biological aggregates (And R H H, Mitchell R. The Role of Polymers in Microbial Aggregation[J]. Annual Review of Microbiology, 1973, 27(1):27-50.), promote the formation of membrane (Sutherland I W. Biofilm Exopolysaccharides[M]. Springer Berlin Heidelberg, 1999.), and provide polymer matrix to form highly hydrated shells for attaining better absorption and accumulation efficiency of nutrients (Decho A W, Herndl G J. Microbial Activities and the Transformation of Organic Matter within Mucilaginous Material[J]. Science of the Total Environment, 1995, 165(1):33-42; Flemming H C, Wingender J. Relevance of Microbial Extracellular Polymeric Substances (EPSs)—Part I: Structural and Ecological Aspects.[J]. Water Science & Technology, A Journal of the International Association on Water Pollution Research, 2001, 43(6):9-16). It is also required for forming a protective layer against potentially toxicological microbial digestion and bacterial predation (Bitton G, Freihofer V. Influence of Extracellular Polysaccharides On the Toxicity of Copper and Cadmium toward Klebsiella Aerogenes.[J]. Microbial Ecology, 1977, 4(2):119; Jeanthon C, Prieur D. Susceptibility to Heavy Metals and Characterization of Heterotrophic Bacteria Isolated From Two Hydrothermal Vent Polychaete Annelids, Alvinella pompejana and Alvinella caudata. [J]. Applied & Environmental Microbiology, 1990, 56(11):3308-3314; Caron D A. Grazing of Attached Bacteria by Heterotrophic Microflagellates[J]. Microbial Ecology, 1987, 13(3):203-218; Decho A W, Lopez G R. Exopolymer Microenvironments of Microbial Flora: Multiple and Interactive Effects On Trophic Relationships[J]. Limnology & Oceanography, 1993, 38(8):1633-1645). Microbial adhesion ability was also mediated by EPS (Paerl H W. Microbial Attachment to Particles in Marine and Freshwater Ecosystems. [J]. Microbial Ecology, 1975, 2(1):73-83.), and among most microbial communities, EPS played a role of antifreezing (Krembs C, Eicken H, Junge K, et al. High Concentrations of Exopolymeric Substances in Arctic Winter Sea Ice: Implications for the Polar Ocean Carbon Cycle and Cryoprotection of Diatoms. [J]. Deep Sea Research Part I: Oceanographic Research Papers, 2002, 49(12):2163-2181; Logan B E, Hunt J R. Advantages to Microbes of Growth in Permeable Aggregates in Marine Systems. [J]. Limnology & Oceanography, 1987, 32(5):1034-1048). Although the production of EPS is extremely energy consuming for microbes (approximately 70% of total cellular energy), its ecological significance enhances the viability and competitiveness of microorganisms in the changing environment (Costerton J W, Ingram J M, Cheng K J. Structure and Function of the Cell Envelope of Gram-Negative Bacteria.[J]. Rev Can Biol, 1970, 38(1):299-316).

Previous studies performed to identify the bioactivity of Stenotrophomonas maltophilia isolated from clinical and environment sources. The isolates from Stenotrophomonas maltophilia were characterized by in vitro antagonism against pathogenic fungi and the production of antifungal metabolites and enzymes (Minkwitz, A., Berg, G., 2001). Comparison of antifungal activities and 16S ribosomal DNA sequences of clinical and environmental isolates of Stenotrophomonas maltophilia. (J. Clin. Microbiol. 39(1), 139-145).

### SUMMARY OF THE INVENTION

The invention herein describes a method for identification of the gliding behaviour of Stenotrophomonas maltophilia, strain P3-15 in different culture conditions and its associated metabolites production.

In view of the foregoing, an embodiment herein provides a method for identification of the gliding behaviour of Stenotrophomonas maltophilia comprises isolation of Stenotrophomonas maltophilia from various ecological niches, growing the S. maltophilia on solid Luria-Bertani (LB) medium supplemented with 100 μg/mL cyclohexamide, 50 μg/mL ampicillin and 25 μg/mL kanamycin, thereafter isolating single colonies formed on solid LB medium and inoculating the single colonies onto selective culture medium selected from 0.1% dry yeast plate and 0.1% chitin plate supplied with the same antibiotics in the corresponding manner, isolating the colonies that survived on both the selective medium and inoculating the colonies onto LB liquid culture to make bacterial stock, followed by extracting the genomic DNA from strains purified from selective culture medium using alkaline lysis technique and verifying the P3-15 strain of Stenotrophomonas maltophilia by identifying 16S rRNA and genetic marker, SmeD gene by sequencing analysis and BLAST assay. Then, a plurality of culture mediums with different carbon sources and nitrogen sources are prepared and inoculated with Stenotrophomonas maltophilia, P3-15 strain and incubating at 28° C. for 5 days. The metabolites produced by Stenotrophomonas maltophilia in each culture medium are harvested and a crude extract is prepared for metabolite assay using high performance liquid chromatography analysis (HPLC). A diversified metabolic profile is observed when gliding motility is restricted and no metabolite is produced when gliding motility is observed.

In another embodiment, the metabolites produced by Stenotrophomonas maltophilia are identified that have anti Staphylococcus aureus activity. In yet another embodiment, Stenotrophomonas maltophilia is isolated from the soil using dilution and direct plating technique comprising the following steps of diluting 1 g of soil in 10 ml of sterile distilled water and from which 100 microliter aliquot is inoculated into 50 mL of non-selective Luria-Bertani (LB) medium and growing overnight at 28 degree Celsius and 200 rpm.

In yet another embodiment, the sequence of 16S rRNA of Stenotrophomonas maltophilia, strain P3-15 is amplified using primers selected from the group comprising Sequence ID no. 2 and Sequence ID no. 3 and the SmeD gene is amplified using primers selected from the group comprising Sequence ID no. 4 and Sequence ID no. 5.

In another embodiment, the plurality of culture mediums are prepared by supplementing 0.1% dry yeast cell agar plates with carbon sources and nitrogen sources, the carbon sources is selected from the group comprising glucose, galactose, glycerol and sucrose and the nitrogen sources is selected from the group comprising tryptone, bacterial peptone, yeast extract and ammonium chloride at the concentration (w/v) of 0.1%, 0.5%, 1% and 2%.

In another embodiment, the metabolites are harvested from culture medium which is a solid culture plate comprising the step of cutting the agar plate into small dices with 0.5 cm×0.5 cm dimensions and submerging the small dices into solvent mixture with ethyl acetate to methanol in 60˜90:40˜10 (v/v) ratio to get an ethyl acetate extract, followed by filtering the ethyl acetate extract to remove insoluble materials preferably agar. The crude extract is prepared by drying the ethyl acetate extract under nitrogen laminar flow and re-dissolving into pure methanol, which is taken at an amount of 1˜5 mL methanol for each culture plate for the HPLC analysis.

In another embodiment, the culture medium comprises 1˜8% of glycerol as carbon source and 0.1˜0.8% of yeast extract as nitrogen source. The metabolites are harvested from the culture medium which is a liquid culture medium comprising the step of collecting cell pellets by centrifuging the liquid culture medium at 7000˜13000 rpm for 3˜10 minutes and rinsed with distilled water, adding 10˜50 ml of acetone into the cell pellets, maintained at room temperature for 8˜20 hours, centrifuging and collecting the supernatant the next day to obtain an acetone extract which is vacuum dried and re-dissolved in 0.5˜5 mL methanol for HPLC analysis.

In another embodiment, the HPLC analysis is performed using a gradient elution program comprising aqueous phase (Phase A) and organic phase (Phase B), the elution program is set as 2˜10% B in A from 0-5 minutes, 3˜15%-50˜90% B in A from 5-15 minutes, 90-100% B in A from 15-20˜25 minutes and 100-2˜10% B in A from 25-30˜40 minutes and eluent is detected under UV detector with wavelengths of 220˜320 nm.

In yet another embodiment, the S. maltophilia strain shows high surface gliding motility which moves towards a single direction after forming a circular colony of 6 mm diameter in the culture medium with the concentration of 0.1% dry yeast.

In another embodiment, the gliding motility is retarded in the culture medium with nitrogen sources, the effective retardation is observed in the culture medium with concentration of yeast extract higher than 0.5%, a complete loss of gliding motility in culture medium with 1%-2% tryptone, no visible impact on the gliding motility in the culture medium with peptone and ammonium chloride in the culture medium is inappropriate for the growth of S. maltophilia. The carbon sources supplied in the culture medium with concentration from 0.1% to 2% did not affect the gliding motility of S. maltophilia.

In another embodiment, the metabolite produced in the culture medium supplied with 1˜8% of glycerol as carbon source and 0.1˜0.8% of yeast extract as nitrogen source comprises two major metabolites are produced with anti-Staphylococcus aureus activity in crude extract (5 μl) of the P3-15 strain.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A illustrates the genetic identification of 16S rRNA.

FIG. 1B illustrates the genetic identification of SmeD gene.

FIGS. 2A-2C illustrates the gliding motility observed under the culture conditions comprising 0.1% dry yeast cell as basal composition with 0.5%, 1% and 2% glucose respectively.

FIGS. 3A-3C illustrates the gliding motility observed under the culture conditions comprising 0.1% dry yeast cell as basal composition with 0.5%, 1% and 2% sucrose respectively.

FIGS. 4A-4C illustrates the gliding motility observed under the culture conditions comprising 0.1% dry yeast cell as basal composition with 0.5%, 1% and 2% yeast extract respectively.

FIGS. 5A-5C illustrates the gliding motility observed under the culture conditions comprising 0.1% dry yeast cell as basal composition with 0.5%, 1% and 2% tryptone respectively.

FIG. 6 illustrates the High performance liquid chromatography analysis of metabolites in crude extracts produced by P3-15 in culture media supplied with 1% glucose (top), 1% yeast extract (middle) and 1% tryptone (bottom).

FIG. 7 illustrates the metabolites analysis of P3-15 strain cultured with glycerol and yeast extract used as carbon and nitrogen source respectively.

FIG. 8 illustrates the bioactivity assay of P3-15 strain cultured with glycerol and yeast extract used as carbon and nitrogen source respectively.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein achieve this by providing a method which identifies the gliding behaviour of Stenotrophomonas maltophilia and associated metabolites production.

The Soil samples are collected from coastal harbor area, Tianjin (N: 39.070254°, E:117.281097°). The positioning of the coastal region of Tianjin Da Gang Bay for mining Xanthomonas species where coastal saline soil is affected by nearshore petroleum drilling platform. The soil samples are collected from 5-10 centimeter depth via percussion drilling. The Stenotrophomonas maltophilia is isolated from soil samples using dilution and direct plating method. The dilution is performed by diluting 1 gram of soil sample in 10 mL sterile distilled water. A 100 microliter aliquot is taken from it and inoculated into 50 mL of non-selective Luria-Bertani (LB) medium to grow overnight at 28 degree Celsius, 200 rpm. The next day, a 100 microliter aliquot is taken from the LB medium to plate onto LB solid medium supplemented with 100 μg/mL cyclohexamide, 50 μg/mL ampicillin and 25 μg/mL kanamycin. The single colonies that grew on LB solid medium are directly inoculated onto two selective culture medium containing 0.1% dry yeast cell plate and 0.1% chitin plate supplied with 100 μg/mL cyclohexamide, 50 μg/mL ampicillin and 25 μg/mL kanamycin respectively. The colonies that survived on both the selective culture medium are inoculated into LB liquid culture for making bacterial stocks. When compared with traditional culture-based methods for isolating S. maltophilia strains, the use of selective medium with unusual carbon sources facilitates the rapid enrichment of S. maltophilia strains.

The capability of some Xanthomonas strains to secrete high efficacy lytic enzymes, particularly those associated with the degradation of oligomeric substances, led to polymeric carbon sources to enrich Xanthomonas strains. The solid culture medium with 0.1% dry yeast cell and 0.1% chitin is chosen as selection media. The natural occurrence of Xanthomonas family in soil samples with high organic pollutant content led to unusual degradation capacity of Xanthomonas family on oligomeric carbon sources will enable them to survive the selection of these two media supplemented with appropriate antibiotics. The prey activity of S. maltophilia on these two substrates are observed on both types of selection medium, with small transparent ring immediately surrounding the colony-forming units. This suggests the degradation of oligomeric carbon sources within dry yeast cell and chitin medium.

The genomic DNA is extracted from 12 candidate strains purified from the selective culture medium using alkaline lysis approach. To verify the identity of these bacterial strains, both 16S rRNA and a specific gene marker SmeD gene for identifying Stenotrophomonas maltophilia strains are amplified using Phanta Max Super-Fidelity DNA polymerase (Vazyme Biotech, Norway). Within 150 single colonies that grew from the selective solid culture medium, 130 were Stenotrophomonas species and the other 20 were Pseudomonas species based on 16S rRNA sequencing analysis. This suggests the selection approach being highly efficient for the enrichment of Stenotrophomonas strains. The continued molecular identification approach using specific efflux pump coding gene as biomarker (SmeD) suggests all Stenotrophomonas species being Stenotrophomonas maltophilia.

A specific primer set is designed that targets the 16S rRNA of Xanthomonas family for PCR screening (Yin, Hu, “Detection Methods for the Genus Lysobacter and the Species Lysobacter enzymogenes” (2010). Dissertations and Theses in Biological Sciences. 15). The positive PCR amplicons of 1250 bp for 16S rRNA and amplicons of 180 bp for a species-specific biomarker SmeD gene confirmed the identity of Stenotrophomonas maltophilia, a gram-negative gliding strain of Xanthomonas family. The 16S rRNA is amplified using primer L4 5′-GAG CCG ACG TCG GAT TAG CTA GTT-3′ with sequence ID no. 2 or primer 1525R (5′-AGGAGGTGATCCAGCC-3′ with a sequence ID no. 3 using PCR programs of initial denature at 95 degree Celsius for 5 minutes, followed by 34 cycles with in cycle denature at 95 degree for 30 seconds, annealing at 65 degree for 30 seconds and elongation at 72 degree for 90 seconds. The SmeD gene is amplified using primer SmeD3: 5′-CCA AGA GCCTTT CCG TCA T-3′ with sequence ID no. 4 or primer smeD5: 5′-TCT CGG ACT TCA GCG TGA C-3′ with a sequence ID no. 5 using PCR programs of initial denature at 95 degree Celsius for 5 minutes, followed by 34 cycles with in cycle denature at 95 degree for 30 seconds, annealing at 55 degree for 30 seconds and elongation at 72 degree for 15 seconds. All PCR products are subjected to sequencing analysis (Huada gene sequencing service) and to a basic local alignment search tool (BLAST) assay for further identification. The sequence of the 16S rRNA is given by the Sequence ID no. 1 which is shown below:

GTGCGGTGCACCAGGCGACGATCCGTAGCTGGTCTGAGAGGATGATCAG
CCACACTGGAACTGAGACACGGTCCAGACTCCTACGGGAGGCAGCAGTG
GGGAATATTGGACAATGGGCGCAAGCCTGATCCAGCCATACCGCGTGGG
TGAAGAAGGCCTTCGGGTTGTAAAGCCCTTTTGTTGGGAAAGAAATCCA
GCCGGCTAATACCTGGTTGGGATGACGGTACCCAAAGAATAAGCACCGG
CTAACTTCGTGCCAGCAGCCGCGGTAATACGAAGGGTGCAAGCGTTACT
CGGAATTACTGGGCGTAAAGCGTGCGTAGGTGGTCGTTTAAGTCCGTTG
TGAAAGCCCTGGGCTCAACCTGGGAACTGCAGTGGATACTGGGCGACTA
GAGTGTGGTAGAGGGTAGCGGAATTCCTGGTGTAGCAGTGAAATGCGTA
GAGATCAGGAGGAACATCCATGGCGAAGGCAGCTACCTGGACCAACACT
GACACTGAGGCACGAAAGCGTGGGGAGCAAACAGGATTAGATACCCTGG
TAGTCCACGCCCTAAACGATGCGAACTGGATGTTGGGTGCAATTTGGCA
CGCAGTATCGAAGCTAACGCGTTAAGTTCGCCGCCTGGGGAGTACGGTC
GCAAGACTGAAACTCAAAGGAATTGACGGGGGCCCGCACAAGCGGTGGA
GTATGTGGTTTAATTCGATGCAACGCGAAGAACCTTACCTGGCCTTGAC
ATGTCGAGAACTTTCCAGAGATGGATTGGTGCCTTCGGGAACTCGAACA
CAGGTGCTGCATGGCTGTCGTCAGCTCGTGTCGTGAGATGTTGGGTTAA
GTCCCGCAACGAGCGCAACCCTTGTCCTTAGTTGCCAGCACGTAATGGT
GGGAACTCTAAGGAGACCGCCGGTGACAAACCGGAGGAGGGTGGGGGAT
GACGTCAAGTCATCATGGCCCTTACGGCCAGGGCTACACACGTACTACA
ATGGTAGGGACAGAGGGCTGCAAGCCGGCGACGGTAAGCCAATCCCAGA
AACCCTATCTCAGTCCCGGATTGGAGTCTGCAACTCGACTCCATGAAGT
CGGAATCGCTAGTATCGCAGATCAGCATTGCTGCGTGAATACGTTCCCG
ACTGTACACACGCCCGTCACACCATGGGAGTTTGTGCACAGAGCAGTAG
CTTAACCTTCCGAGTCCTTGCACGGGTGCGATGACTGGGGTGATCGTAC
AGTAGCGTATCGAGTGCAGCGGGGACACTCCTACTAGATAG

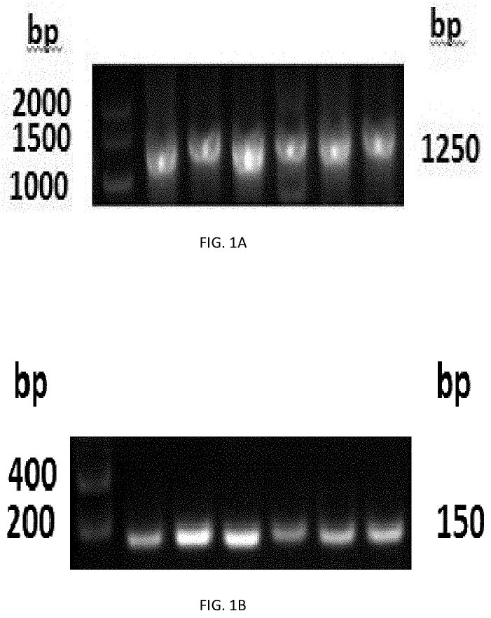

FIG. 1A shows the genetic identification of 16S rRNA identified from the Stenotrophomonas maltophilia strain, P3-15. FIG. 1(B) shows the genetic identification of the presence of a specific gene marker, SmeD gene within the Stenotrophomonas maltophilia strain, P3-15. The S. maltophilia strain, P3-15 is chosen for performing gliding and metabolite assays.

The variation in the culture condition is performed by adding a carbon or a nitrogen source at a concentration ranging from 0.1% (w/v), 0.5% (w/v), 1% (w/v) and 2% (w/v). A total of four carbon sources including glucose, galactose and glycerol, and a two-carbon carbon source sucrose are tested. For nitrogen sources, an inorganic nitrogen source like ammonium chloride and several organic nitrogen sources like tryptone, bacterial peptone and yeast extract are included for metabolite assay. A total of four solid culture plates are prepared for each assay performed for both carbon and nitrogen sources, two of which are served to track the gliding behaviour and the other two served for total metabolite analysis. All the culture plates are incubated at 28° C. for 5 days. The total metabolites are harvested by cutting agar plate into small dices with 0.5 cm×0.5 cm dimensions and submerged in solvent mixture with ethyl acetate to methanol in 60˜90:40˜10 (v/v) ratio. The ethyl acetate extract is then filtered to remove the insoluble materials which is mostly agar and then dried under nitrogen laminar flow and re-dissolved in pure methanol to obtain a crude extract. For each culture plate, the crude extract is dissolved in 1˜5 mL methanol for HPLC analysis with Agilent Technology, Infinity 1260.

The gliding assay is performed to detect the gliding motility of S. maltophilia upon the variation in the culture conditions. The S. maltophilia strain P3-15 are observed to move towards a single direction after forming a circular colony with approximately 6 mm diameter on 0.1% dry yeast cell solid medium, indicating an unusually high surface motility. To examine the impacts of adding different carbon and nitrogen sources on the gliding behaviour, cultures with various carbon and nitrogen sources with increasing concentration are applied.

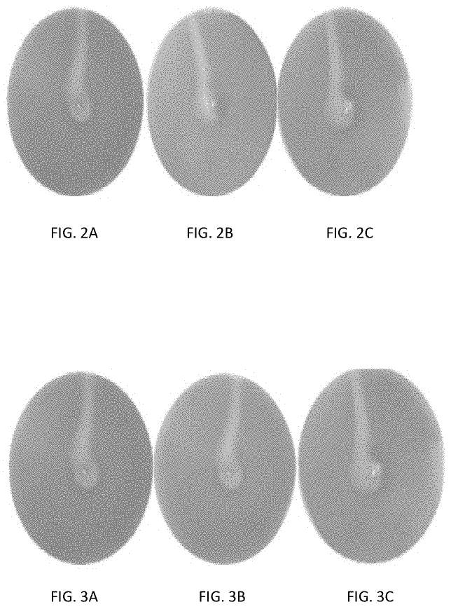

FIGS. 2A-2C illustrates the gliding motility observed under culture conditions with 0.1% dry yeast cell as basal composition with 0.5%, 1% and 2% glucose. The FIGS. 3A-3C illustrates the gliding motility observed under culture conditions with 0.1% dry yeast cell as basal composition with 0.5%, 1% and 2% sucrose. These culture conditions with varying concentration of carbon sources like glucose and sucrose did not make any visible impact on the gliding mobility of S. maltophilia. Other carbon sources used includes galactose and glycerol, therefore all the carbon sources used in the experiment with the concentration from 0.1% to 2% did not affect the gliding behaviour which suggests that change in the carbon source did not make any visible impact on the gliding motility of S. maltophilia.

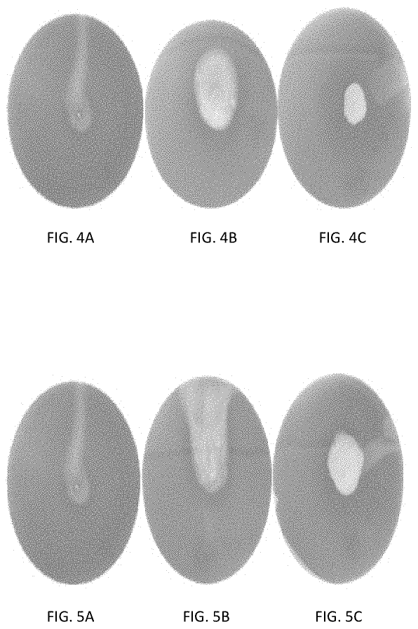

The FIGS. 4A-4C illustrates the gliding motility observed under culture conditions with 0.1% dry yeast cell as basal composition with 0.5%, 1% and 2% yeast extract. The most effective retardation in the gliding motility was achieved by using yeast extract with concentration higher than 0.5%. Upon the supplement of 2% yeast extract in the culture medium, the gliding motility is completely ceased. FIGS. 5A-5C illustrates the gliding motility observed under culture conditions with 0.1% dry yeast cell as basal composition with 0.5%, 1% and 2% tryptone. The treatment of high concentration tryptone (1%˜2%), led to the loss of gliding motility of S. maltophilia. Other nitrogen sources used in the experiment includes bacterial peptone and inorganic nitrogen source like ammonium chloride. The treatment with ammonium chloride in culture medium at low concentration (0-0.5%) led to the retarded bacterial growth while at higher doses (1%-2%), complete cease in growth occurred. This suggests that ammonium chloride to be inappropriate nitrogen source for growing S. maltophilia. The treatment with peptone has no visible impact on the gliding behavior of S. maltophilia at all testing concentrations. Therefore, there is a diversified impact on the gliding mobility of S. maltophilia observed upon variation of the nitrogen sources.

From the experimental results of gliding assay, it can be concluded that the type and concentration of organic nitrogen decides the bacterial response and subsequently the change in the intracellular signaling network that eventually affects the pilus biosynthesis essential for gliding.

Other experiment performed in the culture condition with agar concentration ranging from 1.5% to 1.8% did not reveal any visible change in the gliding behaviour, which suggests that gliding is more of a type of response towards external nitrogen availability

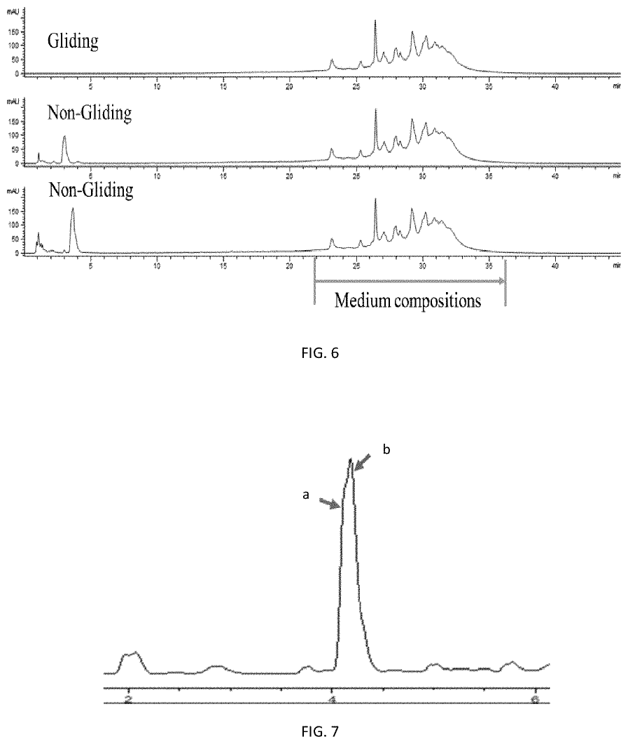

FIG. 6 is the high performance liquid chromatography analysis of the total metabolites in crude extracts produced by S. maltophilia, P3-15 strain. In culture medium supplied with 1% glucose (top), the gliding mobility of P3-15 strain is observed but no metabolite production is detected since there is no peak formation in the chromatogram. In culture medium supplied with 1% yeast extract (middle), there is no gliding mobility observed but a peak is observed indicating the presence of metabolite production. Whereas in the medium supplied with 1% tryptone (bottom), no gliding mobility is observed but a sharp and narrow peak is observed in the chromatogram which represents the metabolites production. A cluster of peaks which is common in all the three chromatogram indicates the medium compositions. Thus, the HPLC analysis suggests that no metabolite is produced in culture medium where the gliding motility is observed. Under the culture conditions where the gliding motility is restricted, a diversified metabolic profile is detected. Therefore, from the above experimental results, it is inferred that the gliding motility is used as a marker to trace culture conditions appropriate for accumulating the desired metabolites.

The high solubility of compounds detected under above non-gliding conditions is likely to result in the failure of column trapping during the course of purification. Therefore, additional fermentation conditions are examined to identify the potential metabolites of S. maltophilia by metabolite assay. The experimental conditions includes the preparation of 500 ml liquid cultures using glycerol (1˜8%) and yeast extract (0.1˜0.8%) as carbon and nitrogen source respectively. The liquid cultures are maintained at 30° C. in a rotatory shaker with 170 rpm for 2 days. To extract the cellular metabolite, cell pellets are collected by centrifuging at 7000˜13000 rpm for 3˜10 minutes and rinsed with distilled water. Then 10˜50 mL acetone are added into the pellet and maintained at room temperature for 8˜20 hours. The supernatant are collected the next day using centrifuge to obtain acetone extract. The acetone extract are then vacuum dried to obtain a crude extract which is re-dissolved in 0.5˜5 mL methanol. Both high performance liquid chromatography (HPLC) and bioactivity analysis are performed to analyze the metabolite chemical and biological properties within the crude extract. The HPLC analysis is performed using a gradient elution program including aqueous phase (Phase A) and organic phase (Phase B). The elution program was set as 2˜10% B in A from 0-5 minutes, 3˜15%-50˜90% B in A from 5-15 minutes, 90-100% B in A from 15-20˜25 minutes and 100-2˜10% B in A from 25-30˜40 minutes. The eluent was detected under UV detector with wavelengths of 220˜320 nm representing the absorbance of aromatic, macrolactam and phytoene compounds.

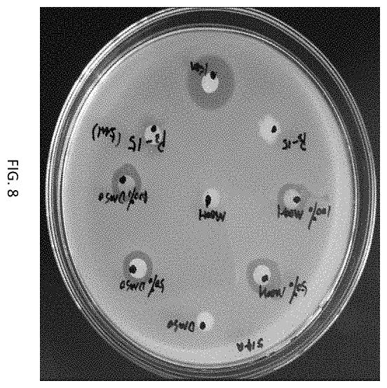

FIG. 7 illustrates the metabolic analysis of crude extract of P3-15 strain cultured with glycerol as carbon and yeast extract as nitrogen source performed using High Performance Liquid Chromatography. The chromatogram shows a sharp peak in which a and b arrows indicate the major metabolites produced by P3-15 under this culture condition. The HPLC analysis suggests that the strain, P3-15 produced two major metabolites which are not detected in other culture conditions with polarity matching of 65% acetonitrile in water. The medium polarity allows easier purification with column chromatography compared to the above two metabolites produced under non-gliding conditions.

FIG. 8 illustrates the bioactivity assay of the crude extracts of P3-15 strain cultured with glycerol and yeast extract used as carbon and nitrogen source. Additional positive controls includes 50% MeOH, 100% MeOH dissolved anti-Staphylococcus WAP (50% MeOH and 100% MeOH), Kanamycin (Kan), 50% DMSO, 100% DMSO dissolved anti-Staphylococcus WAP (50% DMSO and 100% DMSO). Pure DMSO and methanol are used as negative control (DMSO and MeOH). The direct inoculation of P3-15 strain onto this testing medium reveals no anti-Staphylococcus aureus activity (P3-15). The highest anti-Staphylococcus aureus activity is detected with crude extract of P3-15 (5 μl).

The results of foregoing embodiments show that the coupling of the productivity in natural products with the gliding behavior of S. maltophilia indicates that it contains a metabolic network that is quickly witted towards the change in the external nutrition availability. With the appropriate carbon and nitrogen source provided (glycerol as carbon source and yeast extract as nitrogen source), a visible change in the cell morphology and surface smoothness are observed. The chemical analysis and bioactivity analysis of this P3-15 strain resulted in the presence of multiple metabolites and the detection of anti-Staphylococcus aureus activity suggesting a high metabolic potential of S. maltophilia.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A method for identification of the gliding behaviour of Stenotrophomonas maltophilia, and associated metabolites production, the method comprising steps of:
isolating Stenotrophomonas maltophilia from various ecological niches;
growing the Stenotrophomonas maltophilia on solid Luria-Bertani (LB) medium supplemented with 100 μg/mL cyclohexamide, 50 μg/mL ampicillin and 25 μg/mL kanamycin;
isolating single colonies formed on the solid LB medium;
inoculating the single colonies of Stenotrophomonas maltophilia onto selective culture medium;
the selective culture medium selected from 0.1% dry yeast plate and 0.1% chitin plate supplied with 100 μg/mL cyclohexamide, 50 μg/mL ampicillin and 25 μg/mL kanamycin;
isolating colonies that survived both on 0.1% dry yeast plate and 0.1% chitin plate;
inoculating the colonies into LB liquid culture to make bacterial stock for performing metabolite assay and gliding assay;
extracting genomic DNA from strains purified from selective culture medium using alkaline lysis technique;
verifying the P3-15 strain of Stenotrophomonas maltophilia by identifying 16S rRNA and genetic marker, SmeD gene by sequencing analysis and BLAST assay;
preparing a plurality of culture mediums with different carbon sources and nitrogen sources respectively;
inoculating the Stenotrophomonas maltophilia, P3-15 strain into the culture mediums from the bacterial stock;
incubating the culture mediums at 28° C. for 5 days;
observing the gliding motility of Stenotrophomonas maltophilia in each culture medium for the gliding assay;
harvesting the metabolites produced by Stenotrophomonas maltophilia;
preparing a crude extract of the metabolites produced;
subjecting the crude extract to high performance liquid chromatography analysis (HPLC) for metabolite assay,
a diversified metabolic profile is observed when gliding motility is restricted and no metabolite is produced when gliding motility is observed in S. maltophilia.

2. The method as claimed in claim 1, wherein the harvested metabolites has anti Staphylococcus aureus activity.

3. The method as claimed in claim 1, wherein the Stenotrophomonas maltophilia is isolated from soil using dilution and direct plating technique, the dilution and direct plating technique comprises the steps of diluting 1 g of soil in 10 ml of sterile distilled water and from which 100 microliter aliquot is inoculated into 50 mL of non-selective Luria-Bertani (LB) medium and growing overnight at 28 degree Celsius and 200 rpm.

4. The method as claimed in claim 1, wherein the sequence of 16S rRNA of Stenotrophomonas maltophilia, strain P3-15 is identified by SEQ. ID NO. 1.

5. The method as claimed in claim 1, wherein the 16S rRNA is amplified using primers selected from the group comprising SEQ. ID NO. 2 and SEQ. ID NO. 3.

6. The method as claimed in claim 1, wherein the SmeD gene is amplified using primers selected from the group comprising SEQ. ID NO. 4 and SEQ. ID NO. 5.

7. The method as claimed in claim 1, wherein the plurality of culture mediums are prepared by supplementing 0.1% dry yeast cell agar plates with carbon sources and nitrogen sources, the carbon sources is selected from the group comprising glucose, galactose, glycerol and sucrose and the nitrogen sources is selected from group comprising tryptone, bacterial peptone, yeast extract and ammonium chloride.

8. The method as claimed in claim 7, wherein the carbon sources and nitrogen sources in the culture medium is supplied at concentration (w/v) of 0.1%, 0.5%, 1% and 2%.

9. The method as claimed in claim 1, wherein the metabolites are harvested from the culture medium for metabolite assay, the culture medium is a solid culture plate comprising the step of:
cutting the agar plate into small dices with 0.5 cm×0.5 cm dimensions;
submerging the small dices into solvent mixture with ethyl acetate to methanol in 60˜90:40˜10 (v/v) ratio to get an ethyl acetate extract; and
filtering the ethyl acetate extract to remove insoluble materials preferably agar.

10. The method as claimed in claim 1, wherein the crude extract is prepared by drying the ethyl acetate extract under nitrogen laminar flow and re-dissolving into pure methanol, the pure methanol is taken at an amount of 1˜5 mL methanol for each culture plate for the HPLC analysis.

11. The method as claimed in claim 1, wherein the culture medium comprises 1˜8% of glycerol as carbon source and 0.1˜0.8% of yeast extract as nitrogen source.

12. The method as claimed in claim 1, wherein metabolites are harvested from the culture medium, the culture medium is a liquid culture medium comprising step of:
collecting cell pellets by centrifuging the liquid culture medium at 7000˜13000 rpm for 3˜10 minutes and rinsed with distilled water;
adding 10˜50 ml of acetone into the cell pellets, maintained at room temperature for 8˜20 hours;
centrifuging and collecting the supernatant the next day to obtain an acetone extract; and
the acetone extract is vacuum dried and re-dissolved in 0.5˜5 mL methanol for HPLC analysis.

13. The method as claimed in claim 1, the HPLC analysis is performed using a gradient elution program comprising aqueous phase (Phase A) and organic phase (Phase B), the elution program is set as 2˜10% B in A from 0-5 minutes, 3˜15%-50˜90% B in A from 5-15 minutes, 90-100% B in A from 15-20˜25 minutes and 100-2˜10% B in A from 25-30˜40 minutes and eluent is detected under UV detector with wavelengths of 220˜320 nm.

14. The method as claimed in claim 1, wherein the S. maltophilia strain shows high surface gliding motility, the S. maltophilia moves towards a single direction after forming a circular colony.

15. The method as claimed in claim 14, wherein the circular colony is of 6 mm diameter in the culture medium with the concentration of 0.1% dry yeast

16. The method as claimed in claim 1, wherein the gliding motility is retarded in the culture medium with nitrogen sources, the effective retardation is observed in the culture medium with concentration of yeast extract higher than 0.5%, a complete loss of gliding motility in culture medium with 1%-2% tryptone, and no visible impact on the gliding motility in the culture medium with peptone.

17. The method as claimed in claim 1, wherein the carbon sources in the culture medium with concentration from 0.1% to 2% did not affect the gliding motility of S. maltophilia.

18. The method as claimed in claim 1, wherein the metabolites produced in the culture medium supplied with 1˜8% of glycerol as carbon source and 0.1˜0.8% of yeast extract as nitrogen source comprises two major metabolites produced with anti-Staphylococcus aureus activity in crude extract (5 μl) of the P3-15 strain.


SEQUENCE LISTING (5 sequences)

SEQ ID NO: 1 - RNA, 1013 nt, Stenotrophomonas maltophilia
ggcgggcaccaggcgacgaccgagcggcgagaggagacagccacacggaacgagacacggccagacccacgggaggcagcagggggaaaggacaagggcgcaagccgaccagccaaccgcgggggaagaaggcccggggaaagcccggggaaagaaaccagccggcaaaccgggggagacggacccaaagaaaagcaccggcaaccggccagcagccgcggaaacgaaggggcaagcgaccggaaacgggcgaaagcggcgaggggcgaagccgggaaagcccgggccaaccgggaacgcagggaacgggcgacagaggggagagggagcggaaccgggagcaggaaagcgagagacaggaggaacaccaggcgaaggcagcaccggaccaacacgacacgaggcacgaaagcgggggagcaaacaggaagaacccggagccacgcccaaacgagcgaacggagggggcaaggcacgcagacgaagcaacgcgaagcgccgccggggagacggcgcaagacgaaaccaaaggaagacgggggcccgcacaagcggggagagggaacgagcaacgcgaagaaccaccggccgacagcgagaacccagagaggagggcccgggaaccgaacacagggcgcaggcgcgcagccggcggagaggggaagcccgcaacgagcgcaacccgccaggccagcacgaagggggaaccaaggagaccgccgggacaaaccggaggaggggggggagacgcaagcacaggcccacggccagggcacacacgacacaaggagggacagagggcgcaagccggcgacggaagccaacccagaaacccaccagcccggaggagcgcaaccgacccagaagcggaacgcagacgcagacagcagcgcggaaacgcccgacgacacacgcccgcacaccagggagggcacagagcagagcaaccccgagccgcacggggcgagacgggggacgacagagcgacgaggcagcggggacacccacagaag

SEQ ID NO: 2 - RNA, 24 nt, Artificial sequence
  The sequence is put together from several organisms, show closest identity to Stenotrophomonas maltophilia G2 strain,Stenotrophomonas maltophilia 776 strain, and Stenotrophomonas maltophilia FF111 strain
gagccgacgtcggattagctagtt

SEQ ID NO: 3 - RNA, 16 nt, Artificial sequence
  The sequence is put together from several organisms, show closest identity to Stenotrophomonas maltophilia G2 strain,Stenotrophomonas maltophilia 776 strain, and Stenotrophomonas maltophilia FF111 strain
aggaggtgatccagcc

SEQ ID NO: 4 - RNA, 19 nt, Artificial sequence
  The sequence is put together from several organisms, show closest identity to Stenotrophomonas maltophilia G2 strain,Stenotrophomonas maltophilia 776 strain, and Stenotrophomonas maltophilia FF111 strain
ccaagagcctttccgtcat

SEQ ID NO: 5 - RNA, 19 nt, Artificial sequence
  The sequence is put together from several organisms, show closest identity to Stenotrophomonas maltophilia G2 strain,Stenotrophomonas maltophilia 776 strain, and Stenotrophomonas maltophilia FF111 strain
tctcggacttcagcgtgac